(19) United States
(12) Patent Application Publication
(10) Pub. No.: US 2010/0222680 A1
(43) Pub. Date: Sep. 2, 2010

(54) ULTRASOUND IMAGING APPARATUS, IMAGE PROCESSING APPARATUS, IMAGE PROCESSING METHOD, AND COMPUTER PROGRAM PRODUCT

(75) Inventor: Kenji HAMADA, Otawara-shi (JP)

Correspondence Address: OBLON, SPIVAK, MCCLELLAND MAIER & NEUSTADT, L.L.P., 1940 DUKE STREET, ALEXANDRIA, VA, 22314, US

(73) Assignees: KABUSHIKI KAISHA TOSHIBA, Tokyo (JP); TOSHIBA MEDICAL SYSTEMS CORPORATION, Otawara-shi (JP)

(21) Appl. No.: 12/711,523

(22) Filed: Feb. 24, 2010

(30) Foreign Application Priority Data
Feb. 27, 2009 (JP) 2009-047095

(51) Int. Cl. A61B 8/14; G06K 9/00

(52) U.S. Cl. 600/443; 382/131

(57) ABSTRACT

An image-manipulation receiving unit receives an image manipulation by a user. A viewpoint/mark calculating unit calculates a viewpoint and a display position of a probe mark based on the image manipulation by the user. A mark-notation creating unit creates a probe mark, a front-back distinction mark, a line of indicating position just beneath probe center, a line of indicating scan area, and a quadrangular pyramid mark, as a mark. An image compositing unit then composites a color Doppler image with the marks, and displays them onto a monitor.

### CROSS-REFERENCE TO RELATED APPLICATIONS

This application is based upon and claims the benefit of priority from the prior Japanese Patent Application No. 2009-047095, filed on Feb. 27, 2009; the entire contents of which are incorporated herein by reference.

### BACKGROUND OF THE INVENTION

1. Field of the Invention

The present invention relates to a technology for displaying an image taken by an ultrasound imaging apparatus, such as a color Doppler image.

2. Description of the Related Art

An ultrasound imaging apparatus is configured to display information about velocity, such as velocity in a blood vessel, in color as a color Doppler image (for example, see JP-A 2008-237759 (KOKAI)). Moreover, an ultrasound imaging apparatus is configured to display of a power component of a blood flow by using a three-dimensional image, and to display velocity information about an arbitrary cross section specified by a user by using a three-dimensional image as a color Doppler image.

FIG. 5 is a schematic diagram that depicts an example of Multi Planar Reconstruction (MPR) display of color Doppler images and three-dimensional image display according to a conventional technology. As shown in FIG. 5, according to the MPR display, velocity information display 71 is carried out on three cross sections orthogonal to one another. Although FIG. 5 is shown in black and white, display is performed in color on an actual screen based on the speed of a substance and a state whether a substance approaches a probe or recedes from it.

However, when displaying an arbitrary cross section by color Doppler display by turning a three-dimensional image, the position of a probe is not recognized despite that the display is based on the position of the probe, it is difficult to recognize a direction in which the substance moves.

### SUMMARY OF THE INVENTION

According to one aspect of the present invention, an ultrasound imaging apparatus includes a probe that transmits an ultrasound wave to a subject, and receives an ultrasound echo generated in the subject; a data creating unit that creates three-dimensional image data of the subject from the ultrasound echo received by the probe; a cross-sectional image creating unit that creates a cross-sectional image representing a specific cross section from the three-dimensional image data created by the data creating unit; a mark creating unit that creates a mark that indicates positional relation between the cross-sectional image created by the cross-sectional image creating unit and the probe; and a composite-image display unit that composites and displays the cross-sectional image created by the cross-sectional image creating unit and the mark created by the mark creating unit.

According to another aspect of the present invention, an image processing apparatus includes a cross-sectional image creating unit that creates a cross-sectional image representing a specific cross section from three-dimensional image data of an image of a subject taken by an ultrasound imaging apparatus; a mark creating unit that creates a mark that indicates positional relation between the cross-sectional image created by the cross-sectional image creating unit and a probe; and a composite-image display unit that composites and displays the cross-sectional image created by the cross-sectional image creating unit and the mark created by the mark creating unit.

According to still another aspect of the present invention, an image processing method includes creating a cross-sectional image representing a specific cross section from three-dimensional image data of an image of a subject taken by an ultrasound imaging apparatus; creating a mark that indicates positional relation between the created cross-sectional image and a probe; and compositing and displaying the created cross-sectional image and the created mark.

According to still another aspect of the present invention, includes a computer program product having a computer readable medium including a plurality of instructions that is executable by a computer and for processing an image, wherein the instructions, when executed by a computer, cause the computer to perform: creating a cross-sectional image representing a specific cross section from three-dimensional image data of an image of a subject taken by an ultrasound imaging apparatus; creating a mark that indicates positional relation between the created cross-sectional image and a probe; and compositing and displaying the created cross-sectional image and the created mark.

### BRIEF DESCRIPTION OF THE DRAWINGS

FIG. 1 is a schematic diagram that depicts an example of Multi Planar Reconstruction (MPR) images and a three-dimensional image displayed by an ultrasound diagnosis apparatus according to an embodiment of the present invention;

FIG. 2 is a functional block diagram of a configuration of the ultrasound diagnosis apparatus according to the embodiment;

FIG. 3 is a flowchart of a process procedure of display processing of MPR image and a three-dimensional image performed by the ultrasound diagnosis apparatus according to the embodiment;

FIG. 4 is a flowchart of a process procedure of mark creating processing performed by a control/User Interface (UI) unit according to the embodiment; and

FIG. 5 is a schematic diagram that depicts an example of MPR display of color Doppler images and three-dimensional image display according to a conventional technology.

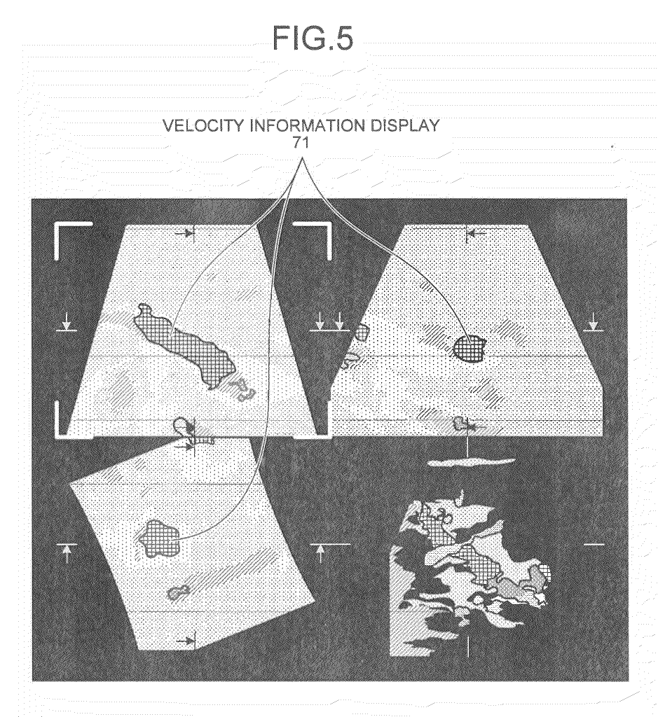

### DETAILED DESCRIPTION OF THE INVENTION

Exemplary embodiments of an ultrasound imaging apparatus, an image processing apparatus, an image processing method, and a computer program product according to the present invention will be explained below in detail with reference to the accompanying drawings.

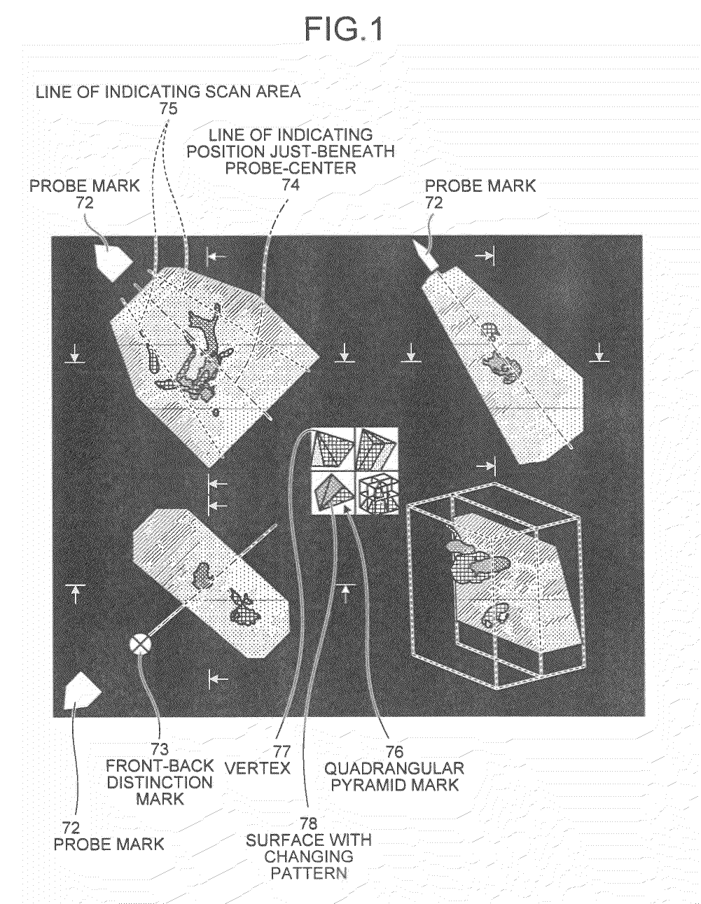

First of all, a Multi Planar Reconstruction (MPR) image and a three-dimensional image displayed by an ultrasound diagnosis apparatus according to an embodiment of the present invention are explained below. FIG. 1 is a schematic diagram that depicts MPR images and a three-dimensional image displayed by the ultrasound diagnosis apparatus according to the embodiment.

As shown in FIG. 1, the ultrasound diagnosis apparatus according to the embodiment displays a probe mark 72 that indicates a direction in which a probe is present on a scale of each color Doppler image displayed by MPR display. When the probe is positioned within a display area, the ultrasound diagnosis apparatus displays the probe mark 72 at the position, and displays a front-back distinction mark that makes a distinction between front and back which the probe is present. In FIG. 1, a front-back distinction mark 73 indicates that the probe is present in front.

Moreover, the ultrasound diagnosis apparatus according to the embodiment deforms the shape of the probe mark 72 in accordance with a direction in which the probe performs a scan. Specifically, when the scanning direction is parallel to the cross section, the width of the probe mark 72 is displayed at the maximum; while the scanning direction is perpendicular to the cross section, the width of the probe mark 72 is displayed at the minimum.

Furthermore, the ultrasound diagnosis apparatus according to the embodiment displays a line of indicating position just-beneath probe-center 74 and a line of indicating scan area 75 on each color Doppler image displayed by MPR display. Moreover, the ultrasound diagnosis apparatus according to the embodiment displays in the center of figure quadrangular pyramid marks 76 each of which indicates relation between a region and a cross section of three-dimensional data and the position of the probe. In FIG. 1, a vertex 77 of the quadrangular pyramid mark 76 indicates the position of the probe, and a surface with changing pattern 78 on the quadrangular pyramid mark 76 indicates a cross section. On an actual image, the quadrangular pyramid mark 76 is to be displayed in different colors instead of different patterns, with respect to a cross section as boundary. Alternatively, the position of the probe can be indicated by a vertex of another pyramid, instead of a quadrangular pyramid. The pyramid is not only the one having a plane bottom, but can be the one having a bottom swelled like a curved surface.

In this way, the ultrasound diagnosis apparatus according to the embodiment can indicate the position and the scan direction of the probe by displaying marks, such as the probe mark 72, the front-back distinction mark 73, the line of indicating position just-beneath probe-center 74, the line of indicating scan area 75, and the quadrangular pyramid mark 76. Accordingly, when displaying velocity information about an arbitrary cross section by turning a three-dimensional image, a direction in which a substance moves can be easily recognized.

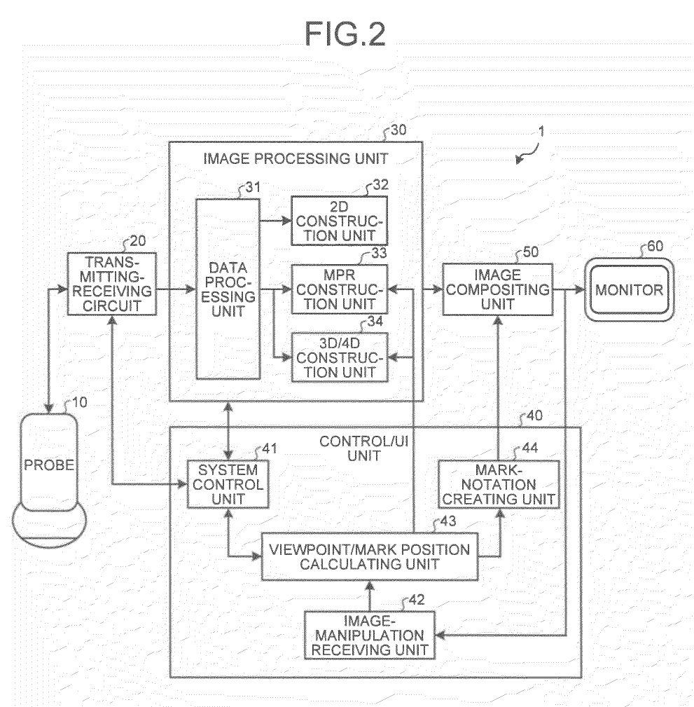

A configuration of the ultrasound diagnosis apparatus according to the embodiment is explained below. FIG. 2 is a functional block diagram of a configuration of the ultrasound diagnosis apparatus according to the embodiment. As shown in FIG. 2, an ultrasound diagnosis apparatus 1 includes a probe 10, a transmitting-receiving circuit 20, an image processing unit 30, a control/User Interface (UI) unit 40, an image compositing unit 50, and a monitor 60.

The probe 10 includes a plurality of ultrasound vibration elements for transmitting and receiving an ultrasound wave, and transmits a transmission signal given as an electric signal by the transmitting-receiving circuit 20 into the subject as an ultrasound wave by using the ultrasound vibration elements. Moreover, the probe 10 receives an ultrasound echo generated in the subject, converts the received ultrasound echo into an echo signal as an electric signal, and passes the converted echo signal to the transmitting-receiving circuit 20.

The transmitting-receiving circuit 20 creates a pulse signal as a transmission signal such that an ultrasound wave is transmitted from the probe 10 in desired transmission timing and with desired transmission intervals, and applies the created transmission signal onto the probe 10. Moreover, the transmitting-receiving circuit 20 acquires an echo signal from the probe 10, and passes the acquired echo signal to the image processing unit 30.

The image processing unit 30 is a processing unit that creates an image from an echo signal, and includes a data processing unit 31, a two-dimensional (2D) construction unit 32, an MPR construction unit 33, and a three-dimensional/four-dimensional (3D/4D) construction unit 34. The data processing unit 31 creates image data, such as a B-mode image, or a color Doppler image, from an echo signal. As a color Doppler image, for example, a velocity component of a substance, a power component, a distribution component, and a high resolution blood flow are displayed.

The 2D construction unit 32 receives image data from the data processing unit 31, and creates a two-dimensional image, such as a B-mode image. The MPR construction unit 33 receives image data from the data processing unit 31, and creates an MPR image from a viewpoint instructed by the control/UI unit 40 with respect to a color Doppler image. The 3D/4D construction unit 34 receives image data from the data processing unit 31, and creates a three-dimensional or four-dimensional image from a viewpoint instructed by the control/UI unit 40.

The control/UI unit 40 is a control unit that controls the ultrasound diagnosis apparatus 1 by receiving an instruction of the user, and includes a system control unit 41, an image-manipulation receiving unit 42, a viewpoint/mark position calculating unit 43, and a mark-notation creating unit 44.

The system control unit 41 controls the whole of the ultrasound diagnosis apparatus. The image-manipulation receiving unit 42 receives an image manipulation by the user, such as a turn of a three-dimensional image. The viewpoint/mark position calculating unit 43 calculates a viewpoint based on a turn operation of a three-dimensional image received by the image-manipulation receiving unit 42, and passes the calculated viewpoint to the MPR construction unit 33 and the 3D/4D construction unit 34. Moreover, the viewpoint/mark position calculating unit 43 calculates the position of the probe on each cross-sectional image, and a display position of the probe mark 72 to be displayed.

The mark-notation creating unit 44 calculates the shape of the probe mark 72 based on the viewpoint and the display position of the probe mark 72 calculated by the viewpoint/mark position calculating unit 43, and creates the probe mark 72. Moreover, the mark-notation creating unit 44 creates the front-back distinction mark 73, the line of indicating position just-beneath probe-center 74, the line of indicating scan area 75, and the quadrangular pyramid mark 76 based on the viewpoint and the display position of the probe mark 72 calculated by the viewpoint/mark position calculating unit 43. The front-back distinction mark 73 and the quadrangular pyramid mark 76 can be individually displayed.

The image compositing unit 50 composites an image created by the image processing unit 30 with a mark created by the mark-notation creating unit 44, and displays them onto the monitor 60. For example, the image compositing unit 50 composites MPR images created by the MPR construction unit 33 with the probe mark 72, the front-back distinction mark 73, the line of indicating position just-beneath probe-center 74, the line of indicating scan area 75, and the quadrangular pyramid mark 76 each created the mark-notation creating unit 44, and a three-dimensional image created by the 3D/4D construction unit 34, and displays them onto the monitor 60.

All or part of the image processing unit 30, the control/UI unit 40, and the image compositing unit 50 can be implemented by application software.

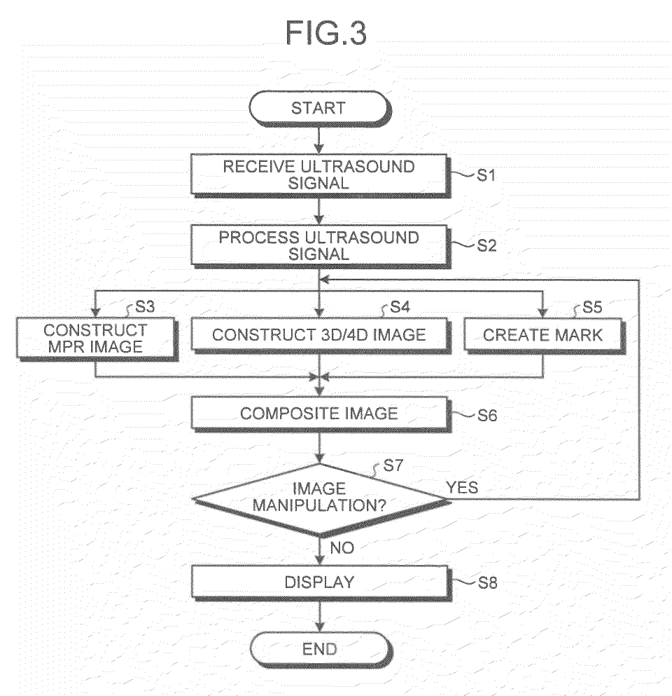

A process procedure of display processing of MPR image and three-dimensional/four-dimensional image performed by the ultrasound diagnosis apparatus 1 according to the embodiment is explained below. FIG. 3 is a flowchart of a process procedure of display processing of MPR images and three-dimensional image performed by the ultrasound diagnosis apparatus 1 according to the embodiment.

As shown in FIG. 3, according to the display processing of MPR image and three-dimensional/four-dimensional image, in the ultrasound diagnosis apparatus 1, the transmitting-receiving circuit 20 receives an ultrasound signal via the probe 10 (Step S1), and the data processing unit 31 creates image data by processing the ultrasound signal (Step S2).

The MPR construction unit 33 then constructs an MPR image (Step S3); the 3D/4D construction unit 34 constructs a three-dimensional image or a four-dimensional image (Step S4); and the control/UI unit 40 creates a mark (Step S5). The processes from Step S3 to Step S5 can be performed in an arbitrary order. Alternatively, the processes can be performed in parallel.

The image compositing unit 50 then composites an image (Step S6), and determines whether an image manipulation is performed on the composited image by the user (Step S7). As a result, if an image manipulation is performed, an MPR image, a three-dimensional image, or a four-dimensional image is reconstructed based on the image manipulation, and mark re-creation is performed. By contrast, if image manipulation is not performed, the image compositing unit 50 displays the composite image (Step S8).

In this way, as the control/UI unit 40 performs mark creation, and the image compositing unit 50 composites the MPR image with the created mark, the position and the scanning direction of the probe 10 can be easily recognized.

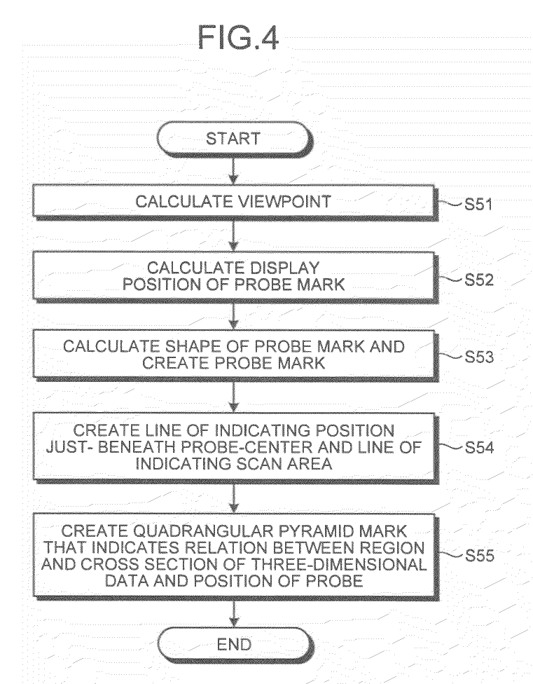

A process procedure of mark creating processing performed by the control/UI unit 40 is explained below. FIG. 4 is a flowchart of a process procedure of mark creating processing performed by the control/UI unit 40 according to the embodiment. The mark creating processing corresponds to the process at Step S5 in FIG. 3.

As shown in FIG. 4, according to the mark creating processing, the viewpoint/mark position calculating unit 43 calculates a viewpoint of an image and a display position of the probe mark 72 based on the image manipulation by the user (Step S51 and Step S52).

The mark-notation creating unit 44 then calculates the shape of the probe mark 72 based on the viewpoint, and creates the probe mark 72 (Step S53). When the probe is positioned in a display area, the mark-notation creating unit 44 creates the front-back distinction mark 73. The mark-notation creating unit 44 then creates the line of indicating position just-beneath probe-center 74 and the line of indicating scan area 75 (Step S54), and creates the quadrangular pyramid mark 76 that indicates relation between the region and the cross section of three-dimensional data and the position of the probe (Step S55).

In this way, as the control/UI unit 40 creates the probe mark 72, the front-back distinction mark 73, the line of indicating position just-beneath probe-center 74, the line of indicating scan area 75, and the quadrangular pyramid mark 76, the position and the scanning direction of the probe 10 can be indicated on the MPR image.

The above process procedure is explained in a case where the control/UI unit 40 creates the probe mark 72, the front-back distinction mark 73, the line of indicating position just-beneath probe-center 74, the line of indicating scan area 75, and the quadrangular pyramid mark 76. However, for example, the control/UI unit 40 can create each of the marks individually.

As described above, according to the embodiment, the image-manipulation receiving unit 42 receives an image manipulation by the user, and the viewpoint/mark position calculating unit 43 calculates the viewpoint and the display position of the probe mark 72 based on the image manipulation by the user. The mark-notation creating unit 44 then creates the probe mark 72, the front-back distinction mark 73, the line of indicating position just-beneath probe-center 74, the line of indicating scan area 75, and the quadrangular pyramid mark 76 as a mark based on the viewpoint and the display position of the probe mark 72. The image compositing unit 50 then composites color Doppler images with the marks, and displays them onto the monitor 60. Accordingly, the position and the scanning direction of the probe 10 can be displayed on MPR display of the color Doppler images, so that a direction in which a substance moves can be easily recognized.

Although the embodiment is explained above in a case of displaying color Doppler images, the present invention is not limited to this, and can be similarly applied to a case of displaying other cross-sectional images.

Moreover, although the embodiment is explained above about the ultrasound diagnosis apparatus, the present invention is not limited this, and similarly applied to an image processing apparatus or an image processing program that acquires image data collected by, such as an ultrasound diagnosis apparatus, and displays velocity information on an image.

As described above, the embodiments of the present invention are suitable for an ultrasound diagnosis apparatus, or an image processing apparatus that extracts velocity information from image data taken by, such as an ultrasound diagnosis apparatus, and displays the extracted information on an image.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound imaging apparatus comprising:
a probe that transmits an ultrasound wave to a subject, and receives an ultrasound echo generated in the subject;
a data creating unit that creates three-dimensional image data of the subject from the ultrasound echo received by the probe;
a cross-sectional image creating unit that creates a cross-sectional image representing a specific cross section from the three-dimensional image data created by the data creating unit;
a mark creating unit that creates a mark that indicates positional relation between the cross-sectional image created by the cross-sectional image creating unit and the probe; and
a composite-image display unit that composites and displays the cross-sectional image created by the cross-sectional image creating unit and the mark created by the mark creating unit.

2. The ultrasound imaging apparatus according to claim 1, further comprising an manipulation receiving unit that receives a manipulation onto the three-dimensional image of the subject specified by a user, wherein
the cross-sectional image creating unit creates the cross-sectional image based on the manipulation received by the manipulation receiving unit, and
the mark creating unit creates the mark based on the manipulation received by the manipulation receiving unit.

3. The ultrasound imaging apparatus according to claim 1, wherein the mark creating unit creates a probe mark that indicates the probe as one of the mark, and deforms a shape of the probe mark based on a scanning direction of the probe.

4. The ultrasound imaging apparatus according to claim 3, the mark creating unit creates a line that indicates a position just beneath a center of the probe as one of the mark.

5. The ultrasound imaging apparatus according to claim 1, wherein the mark creating unit creates a pyramid mark that indicates a position of the probe with a vertex of a pyramid as one of the mark.

6. The ultrasound imaging apparatus according to claim 1, wherein the mark creating unit creates a front-back distinction mark that makes a distinction between front and back which the probe is present.

7. An image processing apparatus comprising:
a cross-sectional image creating unit that creates a cross-sectional image representing a specific cross section from three-dimensional image data of an image of a subject taken by an ultrasound imaging apparatus;
a mark creating unit that creates a mark that indicates positional relation between the cross-sectional image created by the cross-sectional image creating unit and a probe; and
a composite-image display unit that composites and displays the cross-sectional image created by the cross-sectional image creating unit and the mark created by the mark creating unit.

8. An image processing method comprising:
creating a cross-sectional image representing a specific cross section from three-dimensional image data of an image of a subject taken by an ultrasound imaging apparatus;
creating a mark that indicates positional relation between the created cross-sectional image and a probe; and
compositing and displaying the created cross-sectional image and the created mark.

9. A computer program product having a computer readable medium including a plurality of instructions that is executable by a computer and for processing an image, wherein the instructions, when executed by a computer, cause the computer to perform:
creating a cross-sectional image representing a specific cross section from three-dimensional image data of an image of a subject taken by an ultrasound imaging apparatus;
creating a mark that indicates positional relation between the created cross-sectional image and a probe; and
compositing and displaying the created cross-sectional image and the created mark.
